(19) United States
(12) Patent Application Publication
(10) Pub. No.: US 2009/0069738 A1
(43) Pub. Date: Mar. 12, 2009

(54) Electrode Structures Having Anti-Inflammatory Properties And Methods Of Use

(75) Inventors: Martin A. Rossing, Coon Rapids, MN (US); Robert S. Kieval, Medina, MN (US); Mary L. Cole, St. Paul, MN (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: CVRX, Inc., Maple Grove, MN (US)

(21) Appl. No.: 12/174,428

(22) Filed: Jul. 16, 2008

Related U.S. Application Data
(62) Division of application No. 11/322,841, filed on Dec. 29, 2005, now abandoned.

(51) Int. Cl. A61N 1/05

(52) U.S. Cl. 604/20; 607/116; 600/377

(57) ABSTRACT

Electrode structures for implantation include an electrode, an elastic backing attached to the electrode, and a sequestered drug on the backing or the electrode which is released to control and/or limit the growth of scar tissue. The elastic backing includes a tissue contacting side which contacts a tissue structure, and an exposed side away from the tissue contacting side. The electrode structure can be used to activate baroreceptors, and the backing is elastic and can stretch and retract with the tissue structure, for example the carotid sinus. The electrode and the sequestered drug can be positioned on the tissue contacting side so the drug is released near the electrode. The backing can be made from an insulating material and limit diffusion of the drug toward the exposed side, permitting scar tissue to grow near the exposed side to maintain positioning of the electrode.

### CROSS-REFERENCES TO RELATED APPLICATIONS

This application is related to and claims priority as a divisional of U.S. patent application Ser. No. 11/322,841, (Attorney Docket No. 021433-001900US) filed Dec. 29, 2005.

### BACKGROUND OF THE INVENTION

1. Field of the Invention

The present invention relates generally to medical devices and methods for treating heart failure and hypertension. More specifically, the present invention relates to inhibiting an inflammatory response at the site of electrode implantation.

The treatment of a wide variety of conditions can benefit from the use of implantable electrodes. Inflammation caused by the implantation of electrodes can result in the growth of scar tissue. While scar tissue growth can be beneficial in certain circumstances, such as where the scar tissue helps to hold an implanted lead in place or where the scar tissue protects tissues located near an implanted lead. However, the growth of scar tissue can also present undesirable effects where the scar tissue grows between an electrode surface and an underlying tissue which is stimulated with the electrode, as the scar tissue can present a barrier to the stimulation of the underlying tissue. Scar tissue which acts as a barrier to stimulation can reduce the effectiveness of a device implanted to stimulate tissue. Thus, there exists a need to limit or control the growth of scar tissue with at least some implanted electrodes.

Of particular interest to the present invention, certain types of implantable electrodes are designed to be placed over a tissue surface. For example, particular implantable electrode structures disclosed in the co-pending patent applications referenced above comprise a membrane, or backing, which can be wrapped around a carotid sinus or other vascular structure. The backing holds an electrode structure in place over a baroreceptor to permit baroreceptor stimulation to induce the baroreflex to control hypertension or other conditions. The implantation of such electrode structures may result in inflammation as described above with scar formation and other undesirable consequences. Work in connection with the present invention suggests that the mechanical properties of such electrode structures may play a role in the formation of scar tissue. For example, placement of a rigid structure over tissue structures which move frequently, for example an artery, may contribute to scar tissue formation.

For these reasons, it would be desirable to provide improved electrode structures, and methods for their implantation, which result in reduced inflammation. It would be particularly desirable if the electrode structures and implantation methods necessitated minimal changes in present assemblies, designs and implantation protocols. At least some of these objectives will be met by the inventions described below.

2. Description of the Background Art

The following U.S. patents may be relevant to the present application: U.S. Pat. No. 6,522,926; 6,253,110; 6,073,048; 5,987,746; 5,853,652; 5,776,178; 5,766,527; 5,700,282; 5,522,874; 5,408,744; 5,282,844; 5,265,608; 5,092,332; 5,086,787; 4,972,848; 5,991,667; 5,154,182; 5,324,325; 5,154,182; 4,711,251. The following commonly owned patent U.S. applications may be relevant to the present application: Ser. No. 10/284,063 and Ser. No. 11/168231. The following U.S. patent application publications may be relevant to the present application: U.S. 20040062852, 20040010303, 20050182468, 20030060858, 20030060857, 20030060848, 20040010303, 20040019364, 20040254616; PCT patent application publication number WO 99/51286. The full disclosures of the aforementioned patents and applications are herein incorporated by reference.

### BRIEF SUMMARY OF THE INVENTION

The present invention provides electrode structures for implantation into the human body and methods for implanting the electrodes. In particular, the invention provides electrode structures for long term stimulation of baroreceptors located within the wall of blood vessels. Scar tissue formation is inhibited with a combination of an elastic backing and drug, for example an anti-inflammatory substance, eluted or otherwise released near an electrode. The backing which holds the electrode in place over a blood vessel is adapted to stretch, as the blood vessel changes size, thereby minimizing tissue damage. In many embodiments the electrode, for example a coil electrode, is also adapted stretch to minimize tissue damage. The drug is sequestered, on the electrode and/or the backing near the electrode, to minimize inflammation and scar tissue formation.

Electrode structures according to the present invention include an electrode and an elastic backing to hold the electrode in place on a tissue surface. The elastic backing has a tissue contacting side and an exposed side. The elastic backing stretches and changes size with tissue structures, for example a blood vessel, thereby minimize damage to the tissue. A drug, for example a steroid, is positioned to be released to inhibit inflammation in order to control and/or limit the growth of scar tissue around the implanted electrodes, such as electrodes implanted to activate baroreceptors of the carotid sinus. Usually, the drug is sequestered near the electrode to reduce scar tissue formation. The electrode and the drug can be disposed on the tissue contacting side toward the baroreceptors when the backing is placed on or around the carotid sinus or other vascular structure.

In many embodiments, the backing is adapted to stretch while the backing is wrapped at least partially around a pulsating or otherwise tissue structure, such as a blood vessel. For example, the backing can include an elastic, electrically insulating layer which is disposed toward the exposed side of the backing. This elastic, electrically insulating layer can protect tissue near the exposed side from electrical currents. In addition to the electrically insulating layer, the backing can include another sheet or layer, usually also elastic, which has been impregnated with the drug. In some embodiments, the electrode and the drug are disposed on the tissue contacting side to elute the drug toward the electrode. Positioning the electrode and the sequestered drug on the same side ensures that the drug and the electrode are in proximity.

In some embodiments, the drug is sequestered in a coating on or over at least a portion of a surface of the backing and/or the electrode. For example, the coating can be disposed on a side of the backing, such as a drug sputtered on the tissue contacting side of the backing. Coating the backing with the drug ensures that the drug is located near the surface of the backing so that the drug can be effectively delivered to tissue engaged by the surface.

In many embodiments, the drug is sequestered in an adhesive impregnated with the drug, and the adhesive is disposed on at least one of the tissue contacting side and the electrode. Using an eluting adhesive permits many choices as to where the sequestered drug can be positioned. For example, the adhesive can attach the backing to the electrode. Also, the adhesive can be applied to a side of the backing, for example to the tissue contacting side around the electrode. The drug can be any drug which inhibits the growth of scar tissue, for example a steroid. The electrode can be coupled to an implantable pulse generator to deliver the stimulating electrical energy, and the electrode can be in the shape of a flexible coil which moves with the elastomer backing. Some embodiments include at least a second electrode on the tissue contacting side, and the drug is sequestered around the first and second electrodes on the tissue contacting side. Optionally, third, forth and more electrodes could be provided.

In some embodiments the electrode includes a recess, for example a recess inside a wire coil, and the sequestered drug is disposed at least partially within the recess. This configuration can ensure that the sequestered drug is held near the electrode. For example, the electrode can be a coil electrode, and an elastic core impregnated with the drug or containing the drug in a central passage thereof can be disposed at least partially within the coil.

In another aspect the invention is directed to a method for inhibiting inflammation at a tissue surface. An elastic backing is positioned on the tissue surface to immobilize an electrode against the surface, thereby ensuring that the electrode can stimulate the tissue after the electrode has been implanted. An amount of an anti-inflammatory substance is eluted from at least one of the backing and the electrode into the tissue to inhibit inflammation of the tissue and limit scar tissue growth around the electrode. The amount of eluted drug is sufficient to inhibit inflammation of the tissue caused by the electrode.

In many embodiments, the elastic backing is positioned at least partially around a tissue structure, for example a blood vessel such as an artery. When positioned wholly or partially around a blood vessel, the elastic backing will expand and contract with pulsation of the tissue structure. For example, the backing can be positioned at least partially around an artery and the backing can stretch and contract with the artery. The electrode can also be adapted to expand and contract with the tissue structure for example being formed as a coil as discussed below. The elastic backing is typically positioned at least half way around a circumference of the tissue structure (although in some instances because of the irregular cross-sections of the carotid artery and other vessels, the electrode structure assumes a 180 degree or greater arc while extending around less than one-half the vessel perimeter so that the elastic backing remains as positioned on the tissue structure. The elastic backing can include an elastic electrically insulating layer to protect tissue positioned away from the electrode, and the electrode and the drug can be disposed toward the tissue surface in relation to the electrically insulating layer.

### BRIEF DESCRIPTION OF THE DRAWINGS

FIG. 1 is a schematic illustration of the upper torso of a human body showing the major arteries and veins and associated anatomy.

FIG. 2A is a cross sectional schematic illustration of a carotid sinus and baroreceptors within a vascular wall.

FIG. 2B is a schematic illustration of baroreceptors within a vascular wall and the baroreflex system.

FIG. 3. shows an electrode structure in which an electrode is attached to a tissue contacting side of an elastomer backing and a drug sequestered on the backing.

FIG. 4 shows an electrode structure with an electrode and a backing in which a drug impregnated elastomer sheet is positioned on an exposed side of the elastomer backing.

FIG. 4A shows an electrode structure with an electrode and a backing in which the drug impregnated elastomer sheet is positioned on tissue contacting side of the elastomer backing.

FIG. 5 shows an electrode structure having a drug coating on a side of the elastomer backing.

FIG. 5A shows the electrode structure of FIG. 5 implanted near a vessel wall to stimulate baroreceptors.

FIG. 6 shows an electrode structure in which the drug is impregnated in an elastomer tube located within a coil electrode.

FIG. 7 shows an electrode structure in which the drug is impregnated into an elastomer adhesive, and the adhesive is used to adhere the electrode to the elastomer backing.

FIG. 8 shows an electrode structure with steroid impregnated into an elastomer adhesive applied preferentially to specific areas of the elastomer backing.

FIGS. 9A and 9B are schematic illustrations of an implantable extraluminal electrode structure having a backing and a sequestered drug in which the electrode structure electrically induces a baroreceptor signal.

FIGS. 10A-10F are schematic illustrations of various possible arrangements of electrodes around the carotid sinus suitable for combination with the backing and sequestered drug.

FIG. 11 is a schematic illustration of a serpentine shaped electrode with an elastic backing, which permit both the electrode and the backing to stretch with an expanding tissue structure.

FIG. 12 is a schematic illustration of a plurality of electrodes aligned orthogonal to the direction of wrapping around the carotid sinus for extravascular electrical activation.

FIGS. 13-16 are schematic illustrations of various multi-channel electrodes for extravascular electrical activation.

FIG. 17 is a schematic illustration of an extravascular electrical activation device including a tether and an anchor disposed about the carotid sinus and common carotid artery.

FIG. 18 is a schematic illustration of an alternative extravascular electrical activation device including a plurality of ribs and a spine.

FIG. 19 is a schematic illustration of an electrode structure for extravascular electrical activation embodiments.

FIG. 20 illustrates a first exemplary electrode structure having an elastic base and plurality of attachment tabs.

FIG. 21 is a more detailed illustration of the electrode-carrying surface of the electrode structure of FIG. 19.

FIG. 22 is a detailed view of the electrode-carrying surface of an electrode structure similar to that shown in FIG. 20, except that the electrodes have been flattened.

FIG. 23 is an illustration of a further exemplary electrode structure constructed in accordance with the principles of the present invention.

FIG. 24 illustrates the electrode structure of FIG. 23 wrapped around the common carotid artery near the carotid bifurcation.

FIG. 25 illustrates the electrode structure of FIG. 23 wrapped around the internal carotid artery.

FIG. 26 is similar to FIG. 25, but with the carotid bifurcation having a different geometry.

### DETAILED DESCRIPTION OF THE INVENTION

The present invention provides improved electrode structures and methods for implanting such structures against tissue surfaces for stimulating biological tissues such as receptors, nerves, muscles, the spinal cord, and the like. The electrode structures will be adapted for long term, usually permanent, implantation and can be subject to an inflammatory response which can initiate scar tissue formation, as described above. The present invention provides structures and protocols for sequestering steroids and other drugs on the electrode structures so that the drugs will be released into target tissues engaging the electrodes to inhibit inflammation and scar tissue formation. While the electrode structures are particularly described with reference to baroreceptor activation for the control of blood pressure, it will be appreciated that they will also have use in the activation and stimulation of other tissues for other purposes.

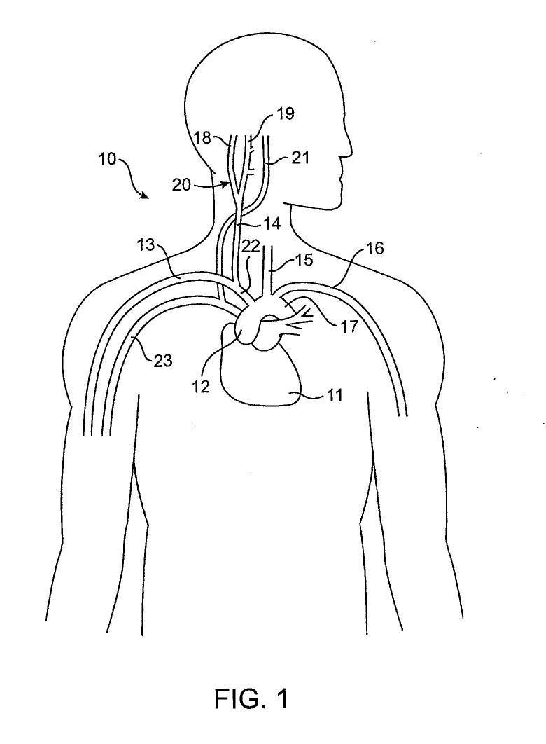

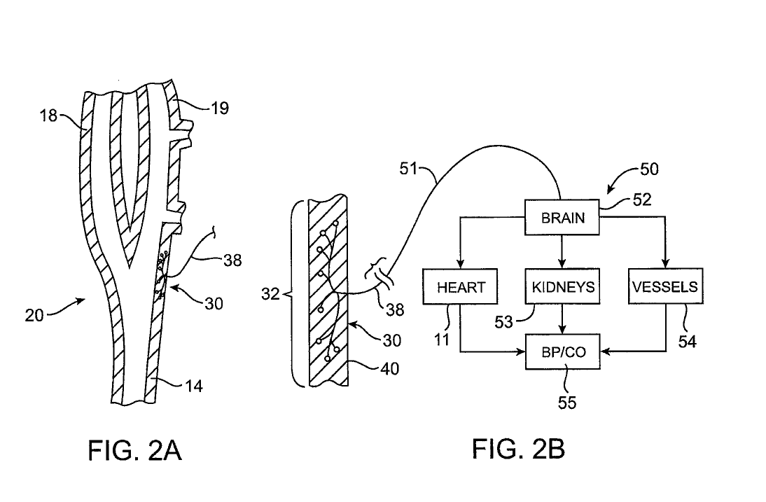

Referring now to FIGS. 1, 2A and 2B, baroreceptors 30 are located within the arterial walls of the aortic arch 12, common carotid arteries 14/15 (near the right carotid sinus 20 and left carotid sinus), subclavian arteries 13/16 and brachiocephalic artery 22. For example, as best seen in FIG. 2A, baroreceptors 30 reside within the vascular walls of the carotid sinus 20. Baroreceptors 30 are a type of stretch receptor used by the body to sense blood pressure. An increase in blood pressure causes the arterial wall to stretch, and a decrease in blood pressure causes the arterial wall to return to its original size. Such a cycle is repeated with each beat of the heart. Baroreceptors 30 located in the right carotid sinus 20, the left carotid sinus, and the aortic arch 12 can play the most significant role in sensing blood pressure that affects baroreflex system 50, which is described in more detail with reference to FIG. 2B.

With reference now to FIG. 2B, baroreceptors 30 are disposed in a generic vascular wall 40 and a schematic flow chart of baroreflex system 50. Baroreceptors 30 are profusely distributed within the arterial walls 40 of the major arteries discussed previously, and generally form an arbor 32. The baroreceptor arbor 32 comprises a plurality of baroreceptors 30, each of which transmits baroreceptor signals to the brain 52 via nerve 38. Baroreceptors 30 are so profusely distributed and arborized within the vascular wall 40 that discrete baroreceptor arbors 32 are not readily discernable. To this end, baroreceptors 30 shown in FIG. 2B are primarily schematic for purposes of illustration.

In addition to baroreceptors, other nervous system tissues are capable of inducing baroreflex activation. For example, baroreflex activation may be achieved in various embodiments by activating one or more baroreceptors, one or more nerves coupled with one or more baroreceptors, a carotid sinus nerve or some combination thereof. Therefore, the phrase “baroreflex activation” generally refers to activation of the baroreflex system by any means, and is not limited to directly activating baroreceptor(s). Although the following description often focuses on baroreflex activation/stimulation and induction of baroreceptor signals, various embodiments of the present invention may alternatively achieve baroreflex activation by activating any other suitable tissue or structure. Thus, the terms “baroreflex activation device” and “baroreflex activation device” are used interchangeably in this application.

Baroreflex signals are used to activate a number of body systems which collectively may be referred to as baroreflex system 50. Baroreceptors 30 are connected to the brain 52 via the nervous system 51, which then activates a number of body systems, including the heart 11, kidneys 53, vessels 54, and other organs/tissues via neurohormonal activity. Such activation of baroreflex system 50 has been the subject of other patent applications by some of the inventors, for example the effect of baroreflex activation on the brain 52 to prevent cardiac arrhythmias and/or promote recovery after occurrence of an arrhythmia. The present methods and apparatus described herein are directed to electrode structures having anti-inflammatory properties which can be used to activate the baroreflex system, ideally for prolonged periods of time.

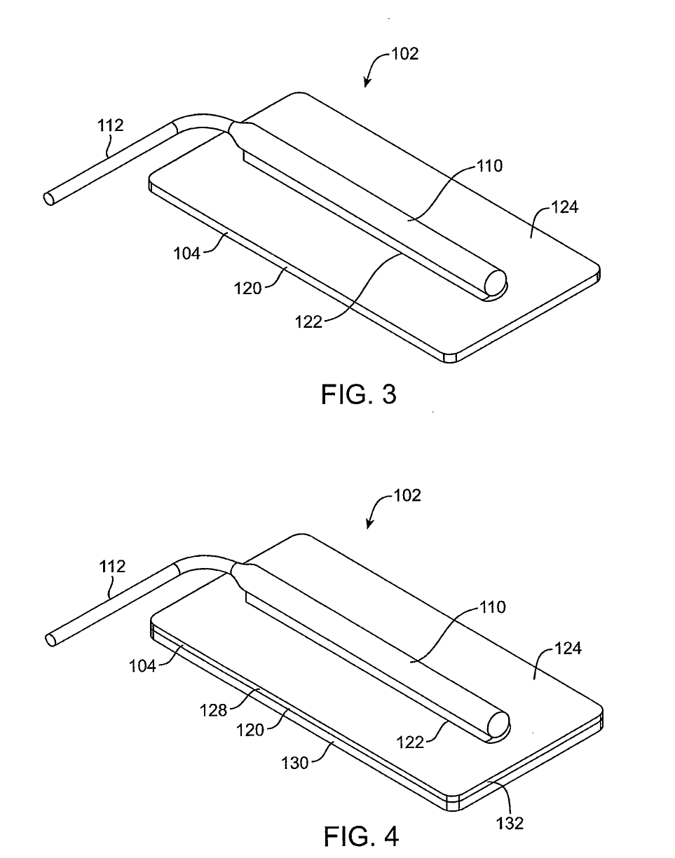

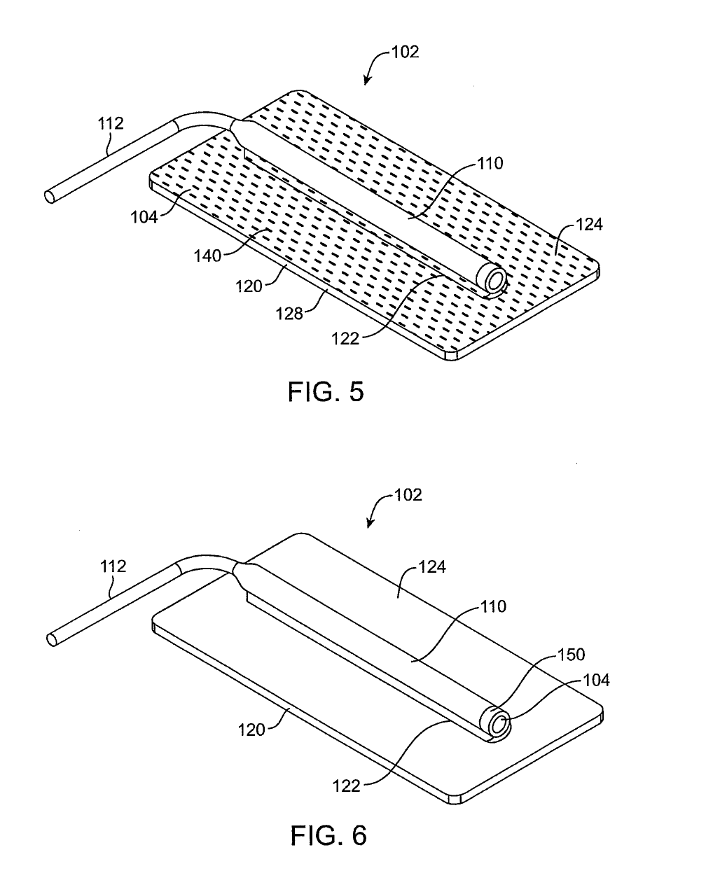

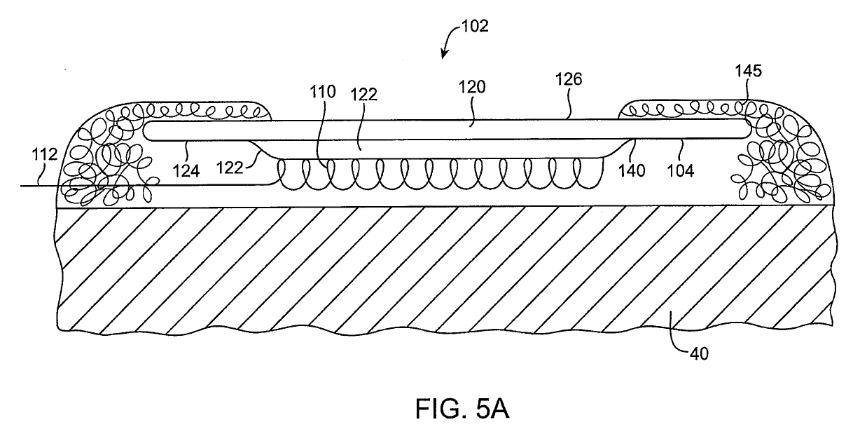

Referring now to the illustration of FIG. 3, an electrode structure 102 includes an electrode 110, and an elastomer backing 120, or backer, and a sequestered drug 104 on the backing. Electrode 110 can have any suitable shape and/or structure, for example a coil, and can be made from any suitable material, for example electrically conducting metals. In preferred embodiments, electrode 110 is a coil of an electrically conducting wire. A coil of wire is desirable because a coil of wire can elastically expand and contract with backing 120, for example while an artery expands and contracts. Other structures such as braided wires and serpentine wires and other electrode structures as described in more detail herein below can also be used to provide electrode structures which separate or stretch with backing 120. Electrode 110 can be attached to an implantable pulse generator (IPG, shown below) with a wire 112. Backing 120 is attached to electrode 110 typically with an adhesive 122. Adhesive 122 can be any suitable adhesive material, for example silicone adhesive. Backing 120 has a tissue contacting side 124, and an exposed side 126 (see FIG. 5A). Backing 120 can be formed from any suitable elastomer or other elastic material which can conform to an underlying tissue structure. For example, backing 120 can expand, or stretch, with the underlying tissue structures, for example arteries. Examples of suitable elastomer materials are silicone materials, for example NuSil silicone rubber which is commercially available. In other embodiments the electrodes could be insert molded into the backing.

Backing 120 can include a variety of materials and several techniques can be used to sequester drug 104 in backing 120. Backing 120 can have electrically insulating properties and be made from any insulating material, for example silicone as described above, to protect tissue near the exposed side of the electrode. In general, backing 120 includes at least one layer of an electrically insulating material. While any suitable electrically insulating material suitable for implantation into the human body can be used, commercially available silicone polymers can be used as an electrically insulating material, for example silicones as described in “Silicones as a Material of Choice for Drug Delivery Applications”, presented Jun. 16, 2004 at the 31st Annual Meeting and Exposition of the Controlled Release Society (http://www.nusil.com/whitepapers/index.aspx). Examples of silicone polymers are also described in “Drug Delivery Market Summary,” published Jun. 25, 2004, (http://www.nusil.com/whitepapers/index.aspx).

Several techniques can be used to sequester drug 104 on backing 120. As shown in FIG. 3, sequestered drug 104 has been impregnated into backing 120. In some embodiments, sequestered drug 104 is coated on an outer surface backing 120 as described herein below. The drug can be any anti-inflammatory substance, and in preferred embodiments is a steroid. Suitable anti-inflammatory drugs include steroids such as dexamethasone acetate, dexamethasone sodium phosphate, prednisone and cortisone, and non-steroidal anti inflammatory drugs (NSAIDs) such as salicylic acid and acetylsalicylic acid, and other anti-hyperplastic drugs such as paclitaxel. The drug can also be any anti-scarring agent as described in U.S. Application Publication No. 2005/0182468, the full disclosure of which has been incorporated by reference above. Techniques for sequestering and eluting drugs are described in U.S. Pat. No. 4,711,251 to Stokes, U.S. Pat. No. 5,522,874 to Gates and U.S. Pat. No. 4,972,848 to Di Domenico et al., the full disclosures of which have been previously incorporated by reference.

Sequestered drug 104 can be included within an electrically insulating layer of backing 120, for example where backing 120 has been impregnated with the drug. Silicone materials impregnated with drugs are available as off the shelf items including silicone materials from NuSil Technology LLC, Carpinteria, Calif. (http://www.nusil.com). In addition to silicone polymers, drug 104 can be sequestered within several other materials. Examples of non-silicone polymers suitable for implantation into the human body in which a drug can be sequestered include styrene isobutylene block copolymers, amino acid-based poly(ester amide) copolymers (PEAs), biodegradable polyesters such as poly(lactic acid)s (PLAs), poly(glycolic acid)s (PGAs) and associated copolymers (PLGAs), poly(anhydride esters) such as “polyNSAIDs” and “polyAsprin” as described in “Polymers Exploited for Drug Delivery”, published in Chemical & Engineering News, Apr. 18, 2005, vol. 83, no. 16, pp. 45-47. Polyurethane, polyurea and/or polyurethane-polyurea can also be employed to sequester drug 104, for example polyurethane and polyurea as described in U.S. Pat. No. 4,972,848, the full disclosure of which has been previously incorporated by reference.

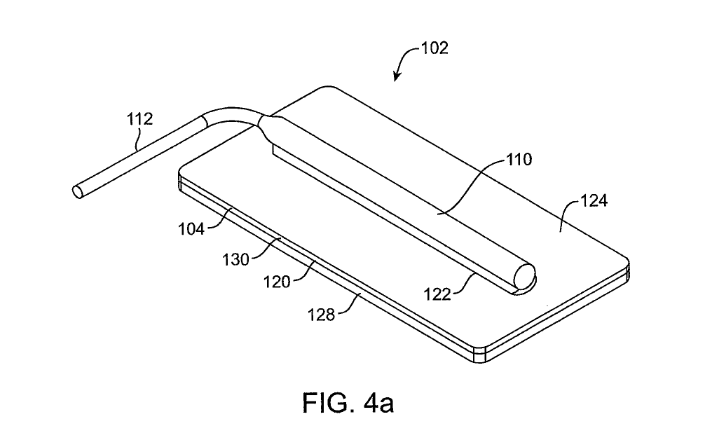

Referring now to the electrode structure illustrated in FIG. 4, sequestration of the drug on the backing can include an elastomer sheet 130 impregnated with the drug and incorporated into backing 120. In this embodiment, backing 120 includes an impregnated elastomer sheet 130 and a non-impregnated elastomer sheet 128. Impregnated sheet 130 can be impregnated with the drug prior to mating impregnated sheet 130 with non-impregnated sheet 128. Impregnated sheet 130 can be laminated to non-impregnated sheet 128 with an adhesive 132 so that sequestered drug 104 is included within backing 120. Impregnated sheet 130 can be laminated to non-impregnated sheet 128 with any suitable adhesive, for example silicone adhesive. As shown in FIG. 4, electrode 110 is located on tissue contacting side 124 of backing 120 and sequestered drug 104 is located on the exposed side of backing 120.

Referring now to the electrode structure illustrated in FIG. 4A, sequestered drug 104 and electrode 110 are positioned on tissue contacting side 124 of backing 120. Sequestered drug 104 is impregnated into sheet 130 as described above. This configuration of electrode structure 102 positions sequestered drug 104 and electrode 110 on the tissue contacting side of electrode structure 102 so that sequestered drug 104 is positioned near electrode 110. Positioning sequestered drug 104 and electrode 110 on the same side of backing 120 can have the advantage of inhibiting the growth of scar tissue near electrode 110 and vessel 40 as described above. At the same time, non-impregnated sheet 128 can decrease diffusion of the drug toward the exposed side of backing 120 so as to permit scar tissue to form on the exposed side and hold the electrode structure in place. This result may be achieved with embodiments in which impregnated sheet 130 and non-impregnated sheet are made from the same polymer, for example silicone. Alternatively, in some embodiments it may be desirable to provide backing 120 in which non-impregnated sheet 128 and impregnated sheet 130 are made from different polymers. For example non-impregnated sheet 128 can be made from an electrically insulating material, such as silicone described above, while impregnated sheet 130 is made from a different silicone or a non-silicone polymer as described above. The potential advantages of providing an electrode structure with electrode 110 and sequestered drug 104 on the same side of the electrode structure are described more fully herein below with reference to FIG. 5A.

Referring now to the electrode structures illustrated in FIGS. 5 and 5A, drug 104 can be sequestered in a coating 140. Backing 120 has tissue contacting side 124 and exposed side 126 as described above. Coating 140 can include the drug and coat tissue contacting side 124 of elastomer backing 120. Following implantation, a scar tissue 145 may form around electrode structure 102 as shown in FIG. 5A. In preferred embodiments, sequestered drug 104 is located near electrode 110 to decrease scar tissue formation between electrode 110 and vessel wall 40 having baroreceptors 30 therein as described above. For example, electrode 110 and coating 140 can be positioned on tissue contacting side 124 of backing 120. As shown in FIGS. 5 and 5A, coating 140 is applied to tissue contacting side 124 near electrode 110 which is also positioned on tissue contacting side 110 to decrease scar tissue formation between electrode 110 and vessel wall 40. Either side or both sides can be coated using techniques used to apply drug coatings to implanted medical devices such as stents and electrodes, for example see U.S. Application Publication No. 20040062852, the full disclosure of which has been previously incorporated by reference. Backing 120 can decrease diffusion of drug molecules from coating 140 toward exposed side 126 of backing 120. Thus, backing 120 can have both electrical insulating properties and chemical insulating properties so as to decrease, at least partially, diffusion of drug molecules to exposed side 126 of backing 120 from coating 140. Consequently, greater amounts of scar tissue 145 may form on exposed side 126 of backing 120 than on tissue contacting side 124 of backing 120.

Referring now to the electrode structure of FIG. 6, the sequestered drug 104 is impregnated in a an elastomer tube 150. Electrode 110 can be a coil of wire having a recess formed thereon. Elastomer tube 150 can be located within the recess formed in electrode 10. Tube 150 can be made from any of the polymers and sequester any of the drugs described above, so that sequestered drug 104 is provided with tube 150. For example, a steroid can be impregnated into elastomer tube 150 so that the steroid is eluted from elastomer tube 150.

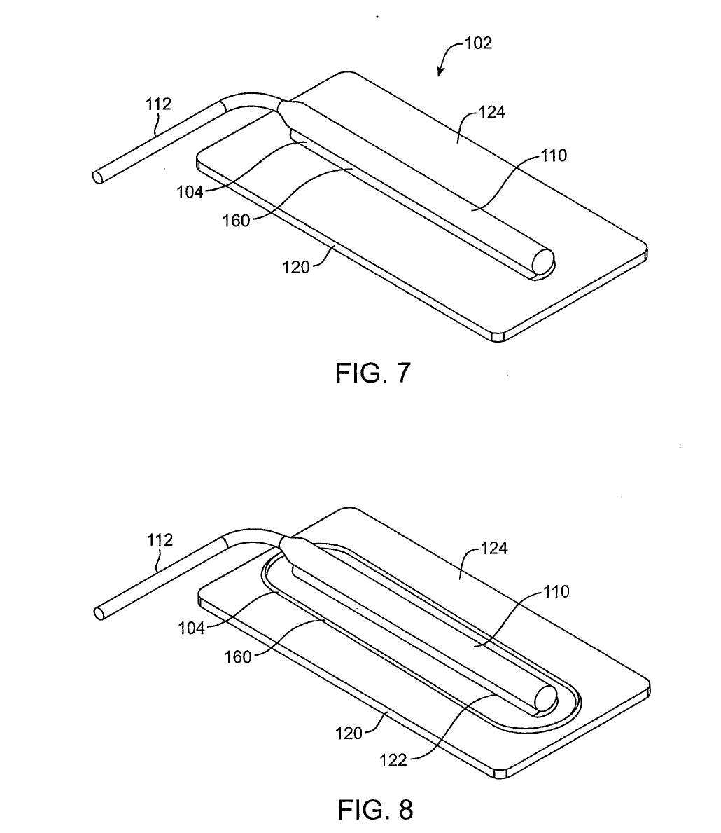

Referring now to the electrode structure illustrated in FIG. 7, drug 104 can be impregnated in an elastomer adhesive 160 to sequester drug 104 in elastomer adhesive 160. Adhesive 160 can be used to adhere electrode 110 to elastomer backing 120. The drug impregnated into elastomer adhesive 160 can be a steroid or other drug as described above.

Referring now to the electrode structure illustrated in FIG. 8, drug impregnated elastomer adhesive 160 can be applied preferentially to specific areas of elastomer backing 120. As shown in FIG. 8, electrode 110 is disposed on tissue contacting side 124 of backing 120, and adhesive 160 has been applied to tissue contacting side 124. Adhesive 160 can be applied around electrode 110 on the tissue contacting side.

The drug eluting structures as described above can be combined with baroreceptor activation systems, electrode geometries, configurations and therapies, for example as described in U.S. application Ser. No. 10/402,911, entitled “Electrode assemblies and methods for their use in cardiovascular reflex control”, published Jan. 15, 2004 as publication number US/20040010303, the full disclosure of which has been previously incorporated by reference. For example, several such electrode configurations and assemblies are described herein below.

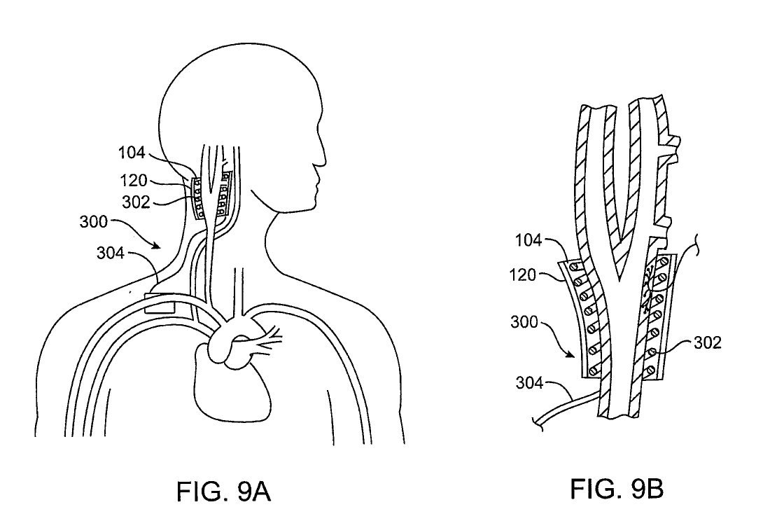

FIGS. 9A and 9B show schematic illustrations of an electrode structure 300 which includes electrodes 302. The structure includes backing 120 and sequestered drug 104 as described above. For example, sequestered drug 104 can be located on the tissue contacting side of backing 120 with electrodes 302, and sequestered drug 104 can be located over the entire surface of the tissue contacting side of backing 120. The electrodes 302 may comprise a coil, braid or other structure capable of surrounding the vascular wall, for example electrode 110 as described above. Alternatively, the electrodes 302 may comprise one or more electrode patches distributed around the outside surface of the vascular wall. Because the electrodes 302 are disposed on the outside surface of the vascular wall, intravascular delivery techniques may not be practical, but minimally invasive surgical techniques will suffice. The extravascular electrodes 302 may receive electrical signals from an implantable pulse generator, or other electrical stimulation device.

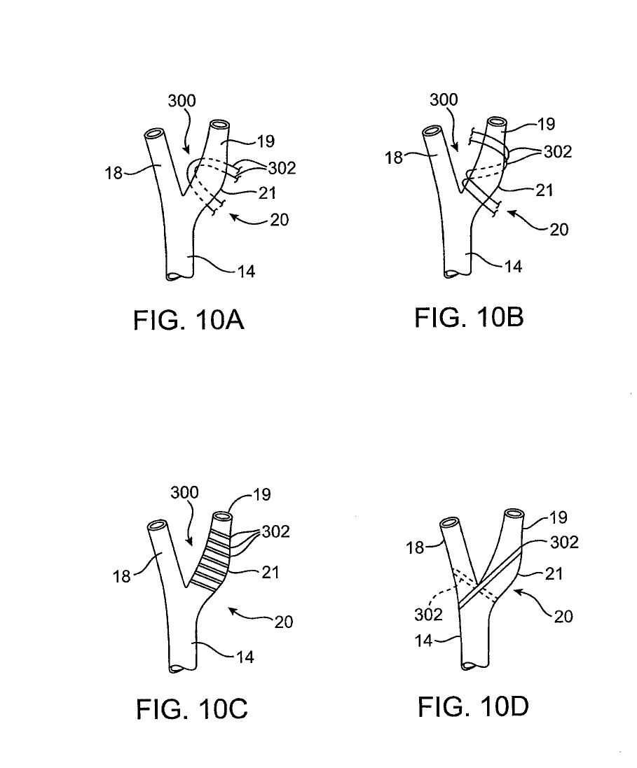

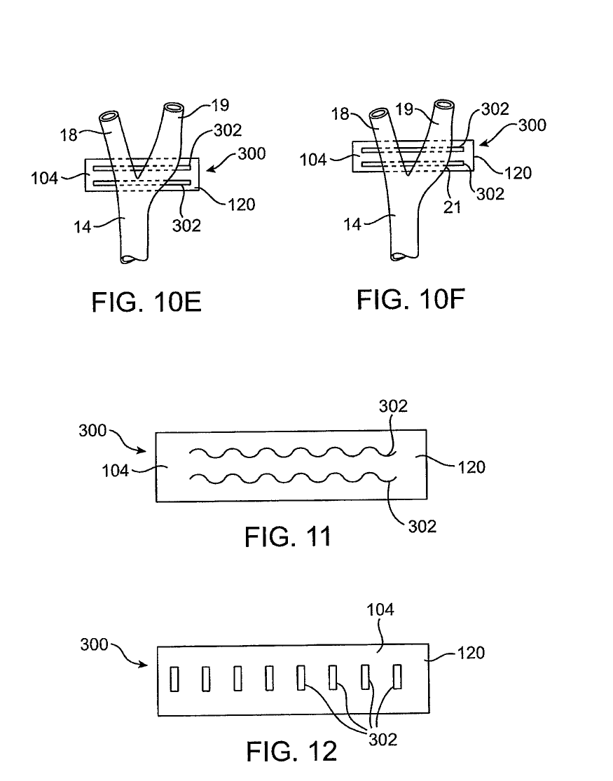

Referring now to FIGS. 10A-10F which show schematic illustrations of various possible arrangements of electrodes around the carotid sinus 20 for extravascular electrical activation embodiments, such as electrode structure 300 described with reference to FIGS. 9A and 9B. The electrodes shown in FIGS. 10A-10F can be combined the backing and sequestered drug on the backing or electrode as described above. For example, the sequestered drug and electrodes can be positioned on the tissue contacting side of the backing as described above. The electrode designs illustrated and described hereinafter may be particularly suitable for connection to the carotid arteries at or near the carotid sinus, and may be designed to minimize extraneous tissue stimulation.

In FIGS. 10A-10F, the carotid arteries are shown, including the common 14, the external 18 and the internal 19 carotid arteries. The location of the carotid sinus 20 may be identified by a landmark bulge 21, which is typically located on the internal carotid artery 19 just distal of the bifurcation, or extends across the bifurcation from the common carotid artery 14 to the internal carotid artery 19.

The carotid sinus 20, and in particular the bulge 21 of the carotid sinus, may contain a relatively high density of baroreceptors 30 (not shown) in the vascular wall. For this reason, it may be desirable to position the electrodes 302 of electrode structure 300 on and/or around the sinus bulge 21 to maximize baroreceptor responsiveness and to minimize extraneous tissue stimulation.

It should be understood that structure 300 and electrodes 302 are merely schematic, and only a portion of which may be shown, for purposes of illustrating various positions of the electrodes 302 on and/or around the carotid sinus 20 and the sinus bulge 21. In each of the embodiments described herein, the electrodes 302 may be monopolar, bipolar, or tripolar (anode-cathode-anode or cathode-anode-cathode sets). Specific extravascular electrode designs are described in more detail hereinafter.

In FIG. 10A, the electrodes 302 of the extravascular electrode structure 300 extend around a portion or the entire circumference of the sinus 20 in a circular fashion. Often, it would be desirable to reverse the illustrated electrode configuration in actual use. In FIG. 10B, the electrodes 302 of the extravascular electrode structure 300 extend around a portion or the entire circumference of the sinus 20 in a helical fashion. In the helical arrangement shown in FIG. 10B, the electrodes 302 may wrap around the sinus 20 any number of times to establish the desired electrode 302 contact and coverage. In the circular arrangement shown in FIG. 10A, a single pair of electrodes 302 may wrap around the sinus 20, or a plurality of electrode pairs 302 may be wrapped around the sinus 20 as shown in FIG. 10C to establish more electrode 302 contact and coverage.

The plurality of electrode pairs 302 may extend from a point proximal of the sinus 20 or bulge 21, to a point distal of the sinus 20 or bulge 21 to ensure activation of baroreceptors 30 throughout the sinus 20 region. The electrodes 302 may be connected to a single channel or multiple channels as discussed in more detail hereinafter. The plurality of electrode pairs 302 may be selectively activated for purposes of targeting a specific area of the sinus 20 to increase baroreceptor responsiveness, or for purposes of reducing the exposure of tissue areas to activation to maintain baroreceptor responsiveness long term.

In FIG. 10D, the electrodes 302 extend around the entire circumference of the sinus 20 in a crisscross fashion. The crisscross arrangement of the electrodes 302 establishes contact with both the internal 19 and external 18 carotid arteries around the carotid sinus 20. Similarly, in FIG. 5E, the electrodes 302 extend around all or a portion of the circumference of the sinus 20, including the internal 19 and external 18 carotid arteries at the bifurcation, and in some instances the common carotid artery 14. In FIG. 10F, the electrodes 302 extend around all or a portion of the circumference of the sinus 20, including the internal 19 and external 18 carotid arteries distal of the bifurcation. In FIGS. 10E and 10F, the extravascular electrode structure 300 are shown to include a backing 120 which may encapsulate and insulate the electrodes 302 and may provide a means for attachment to the sinus 20 as described in more detail hereinafter.

From the foregoing discussion with reference to FIGS. 10A-10F, it should be apparent that there are a number of suitable arrangements for electrodes 302 and elastic backing 120 of the electrode structure 300, relative to the carotid sinus 20 and associated anatomy. In each of the examples given above, electrodes 302 are wrapped around a portion of the carotid structure, which may require deformation of electrodes 302 from their relaxed geometry (e.g., straight). To reduce or eliminate such deformation, the electrodes 302 and/or the backing 306 may have a relaxed geometry that substantially conforms to the shape of the carotid anatomy at the point of attachment. In other words, electrodes 302 and backing 120 may be pre shaped to conform to the carotid anatomy in a substantially relaxed state. Alternatively, the electrodes 302 may have a geometry and/or orientation that reduces the amount of electrode 302 strain. Optionally, as described in more detail below, the base structure or backing 306 may be elastic or stretchable to facilitate wrapping of and conforming to the carotid sinus or other vascular structure.

For example, in FIG. 11, the electrodes 302 are shown to have a serpentine or wavy shape. In a preferred embodiment, the electrodes are located on the tissue contacting side of the backing, and the sequestered drug is located on the tissue contacting side of the backing. For example, the sequestered drug can be located on the tissue contacting side and surround exposed surfaces of the electrodes. The serpentine shape permits the electrode to expand, or stretch with elastic backing 120, for example while an artery pulses. The serpentine shape of the electrodes 302 reduces the amount of strain seen by the electrode material when wrapped around a carotid structure. In addition, the serpentine shape of the electrodes increases the contact surface area of the electrode 302 with the carotid tissue. As an alternative, the electrodes 302 may be arranged to be substantially orthogonal to the wrap direction (i.e., substantially parallel to the axis of the carotid arteries) as shown in FIG. 12. The spacing of the electrodes can separate or contract with elastic backing 120 while an underlying tissue structure such as an artery or vein expands or contracts. In this alternative, the electrodes 302 each have a length and a width or diameter, wherein the length is substantially greater than the width or diameter. The electrodes 302 each have a longitudinal axis parallel to the length thereof, wherein the longitudinal axis is orthogonal to the wrap direction and substantially parallel to the longitudinal axis of the carotid artery about which the structure 300 is wrapped. As with the multiple electrode embodiments described previously, the electrodes 302 may be connected to a single channel or multiple channels as discussed in more detail hereinafter.

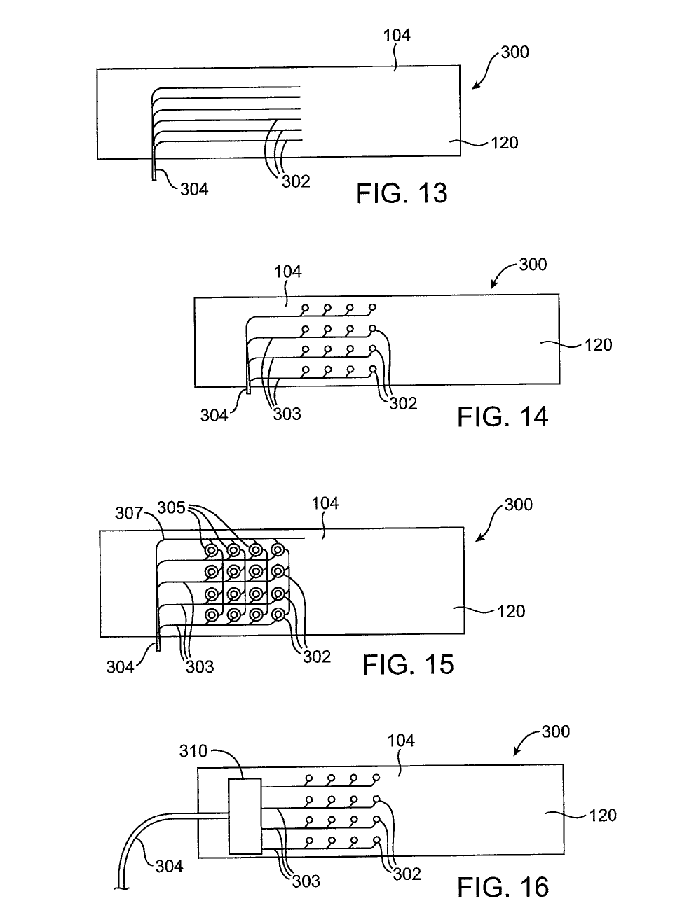

Referring now to FIGS. 13-16 which schematically illustrate various multi-channel electrodes for the extravascular electrode structure 300. Electrode structure 300 generally includes backing 120, sequestered drug 104 and electrodes 302. The sequestered drug and the electrode can be disposed on the same side of the backing, or any other configuration, as described above. FIG. 13 illustrates a six (6) channel electrode structure including six (6) separate elongate electrodes 302 extending adjacent to and parallel with each other. The electrodes 302 are each connected to multi-channel cable 304. Some of the electrodes 302 may be common, thereby reducing the number of conductors necessary in the cable 304.

Backing 120 may comprise a flexible and electrically insulating material suitable for implantation, such as silicone, perhaps reinforced with a flexible material such as polyester fabric as described above. Backing 120 may have a length suitable to wrap around all (360°) or a portion (i.e., less than 360°) of the circumference of one or more of the carotid arteries adjacent the carotid sinus 20. The electrodes 302 may extend around a portion (i.e., less than 360° such as 270°, 180° or 90°) of the circumference of one or more of the carotid arteries adjacent the carotid sinus 20. To this end, the electrodes 302 may have a length that is less than (e.g., 75%, 50% or 25%) the length of the backing 120. The electrodes 302 may be parallel, orthogonal or oblique to the length of backing 120, which is generally orthogonal to the axis of the carotid artery to which it is disposed about. Preferably, the base structure or backing will be elastic (i.e. stretchable), typically being composed of at least in part of silicone, latex, or other elastomer. If such elastic structures are reinforced, the reinforcement should be arranged so that it does not interfere with the ability of the base to stretch and conform to the vascular surface.

The electrodes 302 may comprise round wire, rectangular ribbon or foil formed of an electrically conductive and radiopaque material such as platinum. The backing substantially encapsulates the electrodes 302, leaving only an exposed area for electrical connection to extravascular carotid sinus tissue. For example, each electrode 302 may be partially recessed in the base 206 and may have one side exposed along all or a portion of its length for electrical connection to carotid tissue. Electrical paths through the carotid tissues may be defined by one or more pairs of the elongate electrodes 302.

In all embodiments described with reference to FIGS. 13-16, the multi-channel electrodes 302 may be selectively activated for purposes of mapping and targeting a specific area of the carotid sinus 20 to determine the best combination of electrodes 302 (e.g., individual pair, or groups of pairs) to activate for maximum baroreceptor responsiveness, as described elsewhere herein. In addition, the multi-channel electrodes 302 may be selectively activated for purposes of reducing the exposure of tissue areas to activation to maintain long term efficacy as described, as described elsewhere herein. For these purposes, it may be useful to utilize more than two (2) electrode channels. Alternatively, the electrodes 302 may be connected to a single channel whereby baroreceptors are uniformly activated throughout the sinus 20 region.

An alternative multi-channel electrode design is illustrated in FIG. 14. In this embodiment, electrode structure 300 includes sixteen (16) individual electrodes 302 formed as pads connected to 16 channel cable 304 via 4 channel connectors 303. In this embodiment, the circular electrode pads are partially encapsulated by backing 120 to leave one face of each button of electrodes 302 exposed for electrical connection to carotid tissues. With this arrangement, electrical paths through the carotid tissues may be defined by one or more pairs (bipolar) or groups (tripolar) of the pads.

A variation of the multi-channel pad type electrode design is illustrated in FIG. 15. In this embodiment, electrode structure 300 includes sixteen (16) individual circular pad electrodes 302 surrounded by sixteen (16) rings 305, which collectively may be referred to as concentric electrode pads 302/305. Pad electrodes 302 are connected to 17 channel cable 304 via 4 channel connectors 303, and rings 305 are commonly connected to 17 channel cable 304 via a single channel connector 307. In this embodiment, the circular shaped electrodes 302 and the rings 305 are partially encapsulated by the backing 120 to leave one face of each pad of electrodes 302 and one side of each ring 305 exposed for electrical connection to carotid tissues. As an alternative, two rings 305 may surround each of electrodes 302, with the rings 305 being commonly connected. With these arrangements, electrical paths through the carotid tissues may be defined between one or more pad of electrode 302/ring 305 sets to create localized electrical paths.

Another variation of the multi-channel pad electrode design is illustrated in FIG. 16. In this embodiment, the electrode structure 300 includes a control IC chip 310 connected to 3 channel cable 304. The chip can be an implantable pulse generator. The control chip 310 is also connected to sixteen (16) individual pad electrodes 302 via 4 channel connectors 303. The control chip 310 permits the number of channels in cable 304 to be reduced by utilizing a coding system. A control system sends a coded control signal which is received by chip 310, as described in U.S. Publication No. 20040010303, the full disclosure of which has been previously incorporated by reference. The chip 310 converts the code and enables or disables selected pairs of electrodes 302 in accordance with the code.

For example, the control signal may comprise a pulse wave form, wherein each pulse includes a different code. The code for each pulse causes the chip 310 to enable one or more pairs of electrodes, and to disable the remaining electrodes. Thus, the pulse is only transmitted to the enabled electrode pair(s) corresponding to the code sent with that pulse. Each subsequent pulse would have a different code than the preceding pulse, such that the chip 310 enables and disables a different set of electrodes 302 corresponding to the different code. Thus, virtually any number of electrode pairs may be selectively activated using control chip 310, without the need for a separate channel in cable 304 for each electrode 302. By reducing the number of channels in cable 304, the size and cost thereof may be reduced.

Optionally, the IC chip 310 may be connected to feedback sensor as described in U.S. Application Publication No. 20040010303, previously incorporated by reference. In addition, one or more of the electrodes 302 may be used as feedback sensors when not enabled for activation. For example, such a feedback sensor electrode may be used to measure or monitor electrical conduction in the vascular wall to provide data analogous to an ECG. Alternatively, such a feedback sensor electrode may be used to sense a change in impedance due to changes in blood volume during a pulse pressure to provide data indicative of heart rate, blood pressure, or other physiologic parameter.

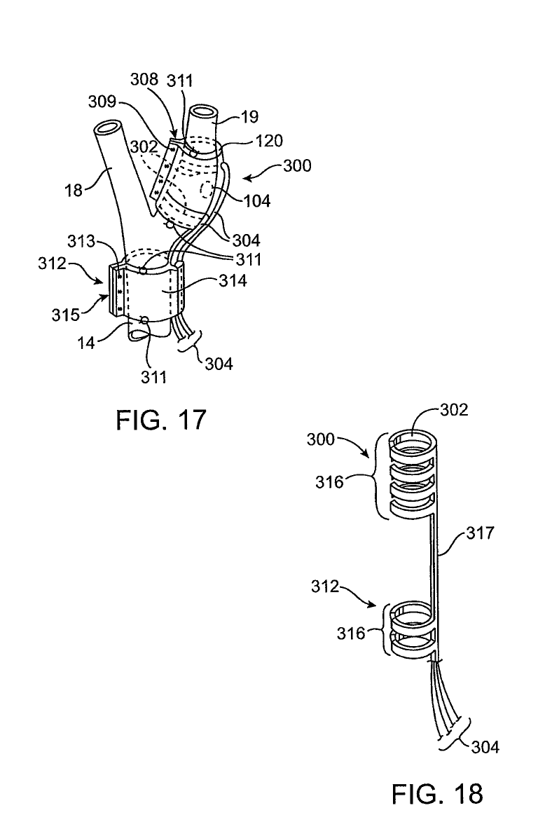

Referring now to FIG. 17 which schematically illustrates an extravascular electrode structure 300 including a support collar or anchor 312. The backing 120 and sequestered drug 104 can be placed in any arrangement as described above. In this embodiment, electrode structure 300 is wrapped around the internal carotid artery 19 at the carotid sinus 20, and the support collar 312 is wrapped around the common carotid artery 14. The electrode structure 300 is connected to the support collar 312 by cables 304, which act as a loose tether. With this arrangement, the collar 312 isolates the activation device from movements and forces transmitted by the cables 304 proximal of the support collar, such as may be encountered by movement of the control system 60 and/or driver 66. As an alternative to support collar 312, a strain relief (not shown) may be connected to baker 306 of electrode structure 300 at the juncture between the cables 304 and the base 306. With either approach, the position of electrode structure 300 relative to the carotid anatomy may be better maintained despite movements of other parts of the system.

In this embodiment, backing 120 of electrode structure 300 may comprise molded tube, a tubular extrusion, or a sheet of material wrapped into a tube shape utilizing a suture flap 308 with sutures 309 as shown. Backing 120 may be formed of a flexible and biocompatible material such as silicone, which may be reinforced with a flexible material such as polyester fabric available under the trade name DACRON® to form a composite structure. The inside diameter of backing 120 may correspond to the outside diameter of the carotid artery at the location of implantation, for example 6 to 8 mm. The wall thickness of backing 120 may be very thin to maintain flexibility and a low profile, for example less than 1 mm. If the structure 300 is to be disposed about a sinus bulge 21, a correspondingly shaped bulge may be formed into the baker for added support and assistance in positioning.

The electrodes 302 (shown in phantom) may comprise round wire, rectangular ribbon or foil, formed of an electrically conductive and radiopaque material such as platinum or platinum iridium. The electrodes may be molded into backing 306 or adhesively connected to the inside diameter thereof, leaving a portion of the electrode exposed for electrical connection to carotid tissues. The electrodes 302 may encompass less than the entire inside circumference (e.g., 300°) of backing 306 to avoid shorting. The electrodes 302 may have any of the shapes and arrangements described previously. For example, as shown in FIG. 12, two rectangular ribbon electrodes 302 may be used, each having a width of 1 mm spaced 1.5 mm apart.

The support collar 312 may be formed similarly to backing 120. For example, the support collar may comprise molded tube, a tubular extrusion, or a sheet of material wrapped into a tube shape utilizing a suture flap 315 with sutures 313 as shown. The support collar 312 may be formed of a flexible and biocompatible material such as silicone, which may be reinforced to form a composite structure. The cables 304 are secured to the support collar 312, leaving slack in the cables 304 between the support collar 312 and electrode structure 300.

In all embodiments described herein, it may be desirable to secure the activation device to the vascular wall using sutures or other fixation means. For example, sutures 311 may be used to maintain the position of electrode structure 300 relative to the carotid anatomy (or other vascular site containing baroreceptors). Such sutures 311 may be connected to backing 120, and pass through all or a portion of the vascular wall. For example, the sutures 311 may be threaded through backing 120, through the adventitia of the vascular wall, and tied. If backing 120 comprises a patch or otherwise partially surrounds the carotid anatomy, the corners and/or ends of the backing may be sutured, with additional sutures evenly distributed therebetween. In order to minimize the propagation of a hole or a tear through backing 120, a reinforcement material such as polyester fabric may be embedded in the silicone material. In addition to sutures, other fixation means may be employed such as staples or a biocompatible adhesive, for example.

Refer now to FIG. 18 which schematically illustrates an alternative extravascular electrode structure 300 including one or more electrode ribs 316 interconnected by spine 317. Optionally, a support collar 312 having one or more (non electrode) ribs 316 may be used to isolate electrode structure 300 from movements and forces transmitted by the cables 304 proximal of the support collar 312.

The ribs 316 of structure 300 are sized to fit about the carotid anatomy, such as the internal carotid artery 19 adjacent the carotid sinus 20. Similarly, the ribs 316 of the support collar 312 may be sized to fit about the carotid anatomy, such as the common carotid artery 14 proximal of the carotid sinus 20. The ribs 316 may be separated, placed on a carotid artery, and closed thereabout to secure structure 300 to the carotid anatomy.

Each of the ribs 316 of structure 300 includes one of electrodes 302 on the inside surface thereof for electrical connection to carotid tissues. The ribs 316 provide insulating material around the electrodes 302, leaving only an inside portion exposed to the vascular wall. The electrodes 302 are coupled to the multi-channel cable 304 through spine 317. Spine 317 also acts as a tether to ribs 316 of the support collar 312, which do not include electrodes since their function is to provide support. The multi-channel electrode 302 functions discussed with reference to FIGS. 8-11 are equally applicable to this embodiment.

The ends of the ribs 316 may be connected (e.g., sutured) after being disposed about a carotid artery, or may remain open as shown. If the ends remain open, the ribs 316 may be formed of a relatively stiff material to ensure a mechanical lock around the carotid artery. For example, the ribs 316 may be formed of polyethylene, polypropylene, PTFE, or other similar insulating and biocompatible material. Alternatively, the ribs 316 may be formed of a metal such as stainless steel or a nickel titanium alloy, as long as the metallic material was electrically isolated from the electrodes 302. As a further alternative, the ribs 316 may comprise an insulating and biocompatible polymeric material with the structural integrity provided by metallic (e.g., stainless steel, nickel titanium alloy, etc.) reinforcement. In this latter alternative, the electrodes 302 may comprise the metallic reinforcement.

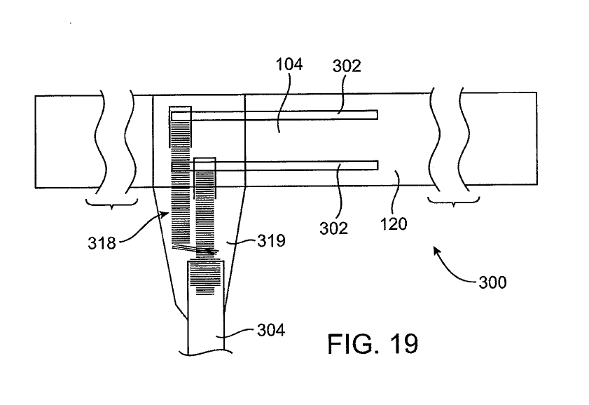

Refer now to FIG. 19 which schematically illustrates a specific example of an electrode structure for an extravascular electrode structure 300. Sequestered drug 104 is located on the tissue contacting side of backing 120, and in this specific example is located on the entire tissue contacting side of backing 120. In this specific example, the backing 120 comprises a silicone sheet having a length of 5.0 inches, a thickness of 0.007 inches, and a width of 0.312 inches. The electrodes 302 comprise platinum ribbon having a length of 0.47 inches, a thickness of 0.0005 inches, and a width of 0.040 inches. The electrodes 302 are adhesively connected to one side of the silicone sheet 306.

The electrodes 302 are connected to a modified bipolar endocardial pacing lead, available under the trade name CONIFIX from Innomedica (now BIOMEC Cardiovascular, Inc.), model number 501112. The proximal end of the cable 304 is connected to the control system 60 or driver 66 as described previously. The pacing lead is modified by removing the pacing electrode to form the cable body 304. The MP35 wires are extracted from the distal end thereof to form two coils 318 positioned side by side having a diameter of about 0.020 inches. The coils 318 are then attached to the electrodes utilizing 316 type stainless steel crimp terminals laser welded to one end of the platinum electrodes 302. The distal end of the cable 304 and the connection between the coils 318 and the ends of the electrodes 302 are encapsulated by silicone.

The cable 304 illustrated in FIG. 19 comprises a coaxial type cable including two coaxially disposed coil leads separated into two separate coils 318 for attachment to the electrodes 302.

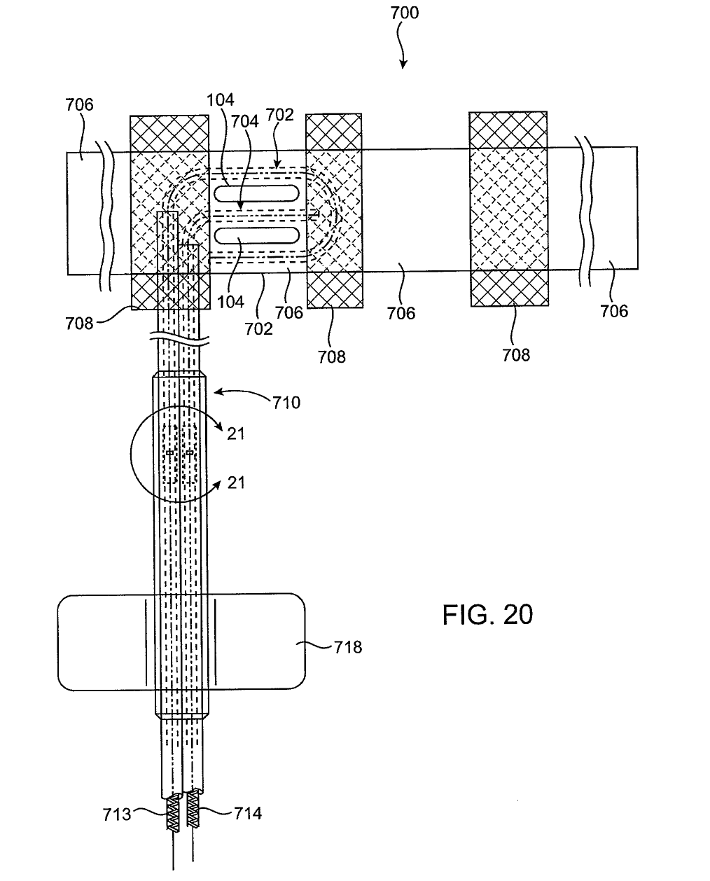

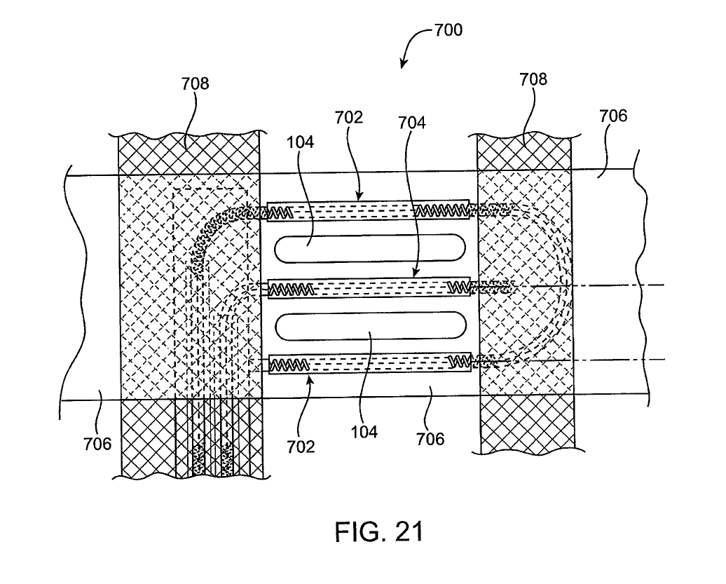

Referring now to FIGS. 20-21 which illustrate an alternative extravascular electrode structure 700. Except as described herein and shown in the drawings, structure 700 may be the same in design and function as electrode structure 300 described previously. Also, sequestered drug 104 can be in any configuration in relation to the backing and electrode as described above.

As seen in FIGS. 20 and 21, electrode cuff structure 700 (or cuff device) includes coiled conducting electrodes 702/704 embedded in a flexible backing 706. Sequestered drug 104 can be disposed on the tissue contacting side of the structure as described above. In the embodiment shown, an outer electrode coil 702 and an inner electrode coil 704 are used to provide a pseudo tripolar arrangement, but other polar arrangements are applicable as well as described previously. In a preferred embodiment, sequestered drug 104 is located between a first portion of outer electrode coil 702 and inner electrode coil 704, and sequestered drug 104 is also located between a second portion of outer electrode coil 702 and inner electrode coil 704, for example. Alternatively, the sequestered drug can be located over the entire tissue contacting side of the backing, or any other configuration as described above. The coiled electrodes 702/704 may be formed of fine round, flat or ellipsoidal wire such as 0.002 inch diameter round PtIr alloy wire wound into a coil form having a nominal diameter of 0.015 inches with a pitch of 0.004 inches, for example. The flexible backing 706 may be formed of a biocompatible and flexible (preferably elastic) material such as silicone or other suitable thin walled elastomeric material having a wall thickness of 0.005 inches and a length (e.g., 2.95 inches) sufficient to surround the carotid sinus, for example.

Each turn of the coil in the contact area of the electrodes 702/704 is exposed from backing 706 and any adhesive to form a conductive path to the artery wall. The exposed electrodes 702/704 may have a length (e.g., 0.236 inches) sufficient to extend around at least a portion of the carotid sinus, for example. The electrode cuff structure 700 is assembled flat with the contact surfaces of the coil electrodes 702/704 tangent to the inside plane of the flexible support 706. When the electrode cuff electrode structure 700 is wrapped around the artery, the inside contact surfaces of the coiled electrodes 702/704 are naturally forced to extend slightly above the adjacent surface of the flexible support, thereby improving contact to the artery wall.

The ratio of the diameter of the coiled electrodes 702/704 to the wire diameter is preferably large enough to allow the coil to bend and elongate without significant bending stress or torsional stress in the wire. Flexibility is a significant advantage of this design which allows the electrode cuff electrode structure 700 to conform to the shape of the carotid artery and sinus, and permits expansion and contraction of the artery or sinus without encountering significant stress or fatigue. In particular, the flexible electrode cuff electrode structure 700 may be wrapped around and stretched to conform to the shape of the carotid sinus and artery during implantation. This may be achieved without collapsing or distorting the shape of the artery and carotid sinus due to the compliance of the cuff electrode structure 700. Backing 706 is able to flex and stretch with the conductor coils 702/704 because of the absence of fabric reinforcement in the electrode contact portion of the cuff electrode structure 700. By conforming to the artery shape, and by the edge of backing 706 sealing against the artery wall, the amount of stray electrical field and extraneous stimulation will likely be reduced.

The pitch of the coil electrodes 702/704 may be greater than the wire diameter in order to provide a space between each turn of the wire to thereby permit bending without necessarily requiring axial elongation thereof. For example, the pitch of the contact coils 702/704 may be 0.004 inches per turn with a 0.002 inch diameter wire, which allows for a 0.002 inch space between the wires in each turn. The inside of the coil may be filled with a flexible adhesive material such as silicone adhesive which may fill the spaces between adjacent wire turns. By filling the small spaces between the adjacent coil turns, the chance of pinching tissue between coil turns is minimized thereby avoiding abrasion to the artery wall. Thus, the embedded coil electrodes 702/704 are mechanically captured and chemically bonded into backing 706. In the unlikely event that a coil electrode 702/704 comes loose from backing 706, the diameter of the coil is large enough to be atraumatic to the artery wall. Preferably, the centerline of the coil electrodes 702/704 lie near the neutral axis of cuff electrode structure 700 and backing 706 comprises a material with isotropic elasticity such as silicone in order to minimize the shear forces on the adhesive bonds between the coil electrodes 702/704 and backing 706.

The electrode coils 702/704 are connected to corresponding conductive coils 712/714, respectively, in an elongate lead 710 which is connected to the control system 60. Anchoring wings 718 may be provided on the lead 710 to tether the lead 710 to adjacent tissue and minimize the effects or relative movement between the lead 710 and the electrode cuff 700. As seen in FIG. 21, the conductive coils 712/714 may be formed of 0.003 MP35N bifilar wires wound into 0.018 inch diameter coils which are electrically connected to electrode coils 702/704 by splice wires 716. The conductive coils 712/714 may be individually covered by an insulating covering 718 such as silicone tubing and collectively covered by insulating covering 720.

The conductive material of the electrodes 702/704 may be a metal as described above or a conductive polymer such as a silicone material filled with metallic particles such as Pt particles. In this latter embodiment, the polymeric electrodes may be integrally formed with backing 706 with the electrode contacts comprising raised areas on the inside surface of backing 706 electrically coupled to the lead 710 by wires or wire coils. The use of polymeric electrodes may be applied to other electrode design embodiments described elsewhere herein.

Reinforcement patches 708 such as DACRON® fabric may be selectively incorporated into backing 706. For example, reinforcement patches 708 may be incorporated into the ends or other areas of backing 706 to accommodate suture anchors. The reinforcement patches 708 provide points where the electrode cuff 700 may be sutured to the vessel wall and may also provide tissue in growth to further anchor the device 700 to the exterior of the vessel wall. For example, the fabric reinforcement patches 708 may extend beyond the edge of backing 706 so that tissue in growth may help anchor the electrode structure or cuff 700 to the vessel wall and may reduce reliance on the sutures to retain the electrode structure 700 in place. As a substitute for or in addition to the sutures and tissue in growth, bioadhesives such as cyanoacrylate may be employed to secure the structure 700 to the vessel wall. In addition, an adhesive incorporating conductive particles such as Pt coated micro spheres may be applied to the exposed inside surfaces of the electrodes 702/704 to enhance electrical conduction to the tissue and possibly limit conduction along one axis to limit extraneous tissue stimulation.

The reinforcement patches 708 may also be incorporated into the flexible support 706 for strain relief purposes and to help retain the coils 702/704 to the backing 706 where the leads 710 attach to the electrode structure 700 as well as where the outer coil 702 loops back around the inner coil 704. Preferably, the patches 708 are selectively incorporated into backing 706 to permit expansion and contraction of the device 700, particularly in the area of the electrodes 702/704. In particular, backing 706 can be only fabric reinforced in selected areas thereby maintaining the ability of the cuff electrode structure 700 to stretch.

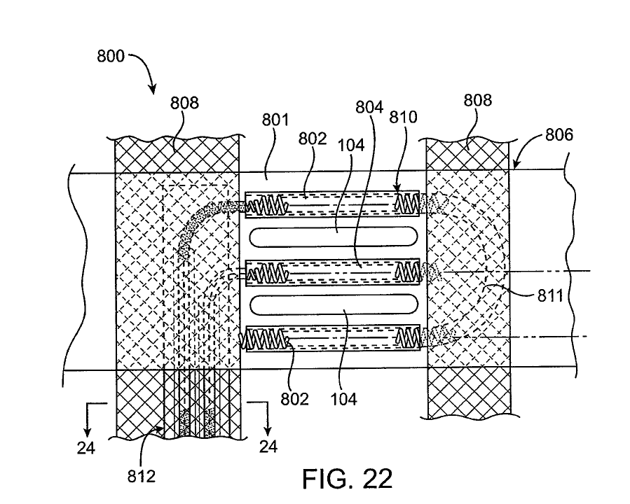

Referring now to an electrode structure 800 shown in FIG. 22, the electrode structure as shown in FIGS. 20-21 can be modified to have “flattened” coil electrodes in the region of the structure where the electrodes contact the extravascular tissue. Sequestered drug 104 can be located relative to the backing in any configuration as described above, including covering the entire tissue contacting side of the backing. In preferred embodiments, sequestered drug 104 is located on the tissue contacting side of the backing between the electrodes as described above. As shown in FIG. 22, an electrode-carrying surface 801 of the electrode structure, is located generally between parallel reinforcement strips or tabs 808. The flattened coil section 810 will generally be exposed on a lower surface of a backing 806 and will be covered or encapsulated by a parylene or other polymeric structure or material 802 over an upper surface thereof. Backing 806 can be similar to backing 120 described above, and generally comprises an elastomeric material as described above. The use of the flattened coil structure is particularly beneficial since it retains flexibility, allowing the electrodes to bend, stretch, and flex together with backing 806, while also increasing the flat electrode area available to contact the extravascular surface.

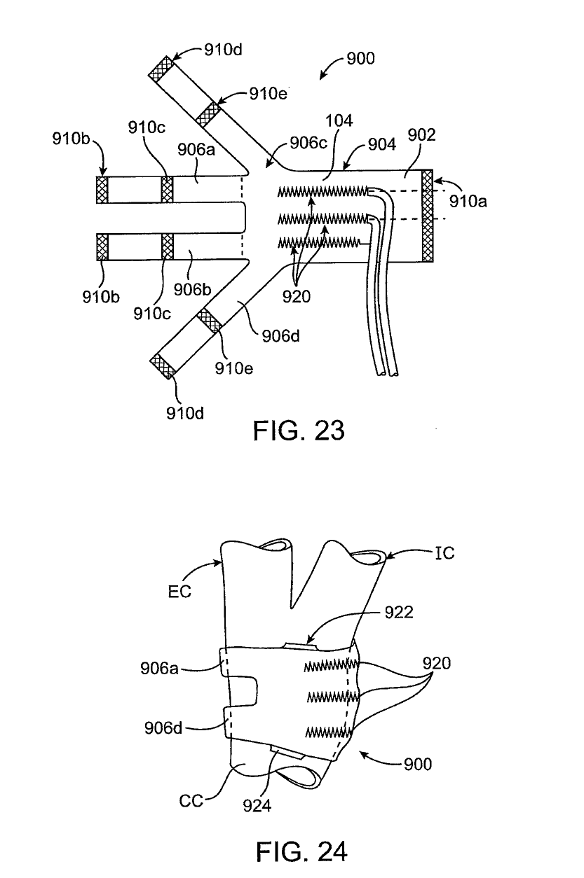

Referring now to FIG. 23, an additional electrode structure 900 will be described. Electrode structure 900 comprises an elastic baking 902, typically formed from silicone or other elastomeric material as described above, having an electrode-carrying surface 904 and a plurality of attachment tabs 906 (906a, 906b, 906c, and 906d) extending from the electrode-carrying surface. Sequestered drug 104 can be positioned on the tissue contacting side of structure 900 as described above, or any other configuration as described above. The attachment tabs 906 are preferably formed from the same material as the electrode-carrying surface 904 of backing 902, but could be formed from other elastomeric materials as well. In the latter case, the backing will be molded, stretched or otherwise assembled from the various pieces. In the illustrated embodiment, the attachment tabs 906 are formed integrally with the remainder of backing 902, i.e., typically being cut from a single sheet of the elastomeric material.

The geometry of the electrode structure 900, and in particular the geometry of the baker 902, is selected to permit a number of different attachment modes to the blood vessel. In particular, the geometry of the structure 900 of FIG. 23 is intended to permit attachment to various locations on the carotid arteries at or near the carotid sinus and carotid bifurcation.

A number of reinforcement regions 910 (910a, 910b, 910c, 910d, and 910e) are attached to different locations on the base 902 to permit suturing, clipping, stapling, or other fastening of the attachment tabs 906 to each other and/or the electrode-carrying surface 904 of backing 902. In the preferred embodiment intended for attachment at or around the carotid sinus, a first reinforcement strip 910a is provided over an end of backing 902 opposite to the end which carries the attachment tabs. Pairs of reinforcement strips 910b and 910c are provided on each of the axially aligned attachment tabs 906a and 906b, while similar pairs of reinforcement strips 910d and 910e are provided on each of the transversely angled attachment tabs 906c and 906d. In the illustrated embodiment, all attachment tabs will be provided on one side of the base, preferably emanating from adjacent corners of the rectangular electrode-carrying surface 904.

The structure of electrode structure 900 permits the surgeon to implant the electrode structure so that the electrodes 920 (which are preferably stretchable, flat-coil electrodes as described in detail above), are located at a preferred location relative to the target baroreceptors. The preferred location may be determined, for example, as described in copending application Ser. No. 09/963,991, filed on Sep. 26, 2001, the full disclosure of which has been previously incorporated herein by reference.

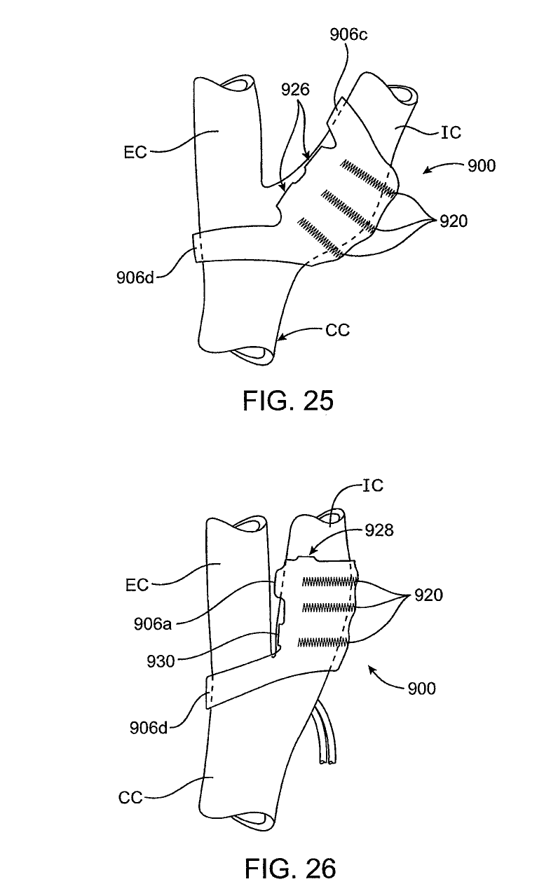

Once the preferred location for the electrodes 920 of the electrode structure 900 is determined, the surgeon may position the base 902 so that the electrodes 920 are located appropriately relative to the underlying baroreceptors. Thus, the electrodes 920 may be positioned over the common carotid artery CC as shown in FIG. 24, or over the internal carotid artery IC, as shown in FIGS. 25 and 26. The external carotid (EC) artery is shown in these figures. In FIG. 28, the structure 900 may be attached by stretching backing 902 and attachment tabs 906a and 906b over the exterior of the common carotid artery. The reinforcement tabs 906a or 906b may then be secured to the reinforcement strip 910a, either by suturing, stapling, fastening, gluing, welding, or other well-known means. Usually, the reinforcement tabs 906c and 906d will be cut off at their bases, as shown at 922 and 924, respectively.

In other cases, the bulge of the carotid sinus and the baroreceptors may be located differently with respect to the carotid bifurcation. For example, as shown in FIG. 25, the receptors may be located further up the internal carotid artery IC so that the placement of electrode structure 900 as shown in FIG. 24 may not work. The structure 900, however, may still be successfully attached by utilizing the transversely angled attachment tabs 906c and 906d rather than the central or axial tabs 906a and 906b. As shown in FIG. 25, the lower tab 906d is wrapped around the common carotid artery CC, while the upper attachment tab 906c is wrapped around the internal carotid artery IC. The axial attachment tabs 906a and 906b will usually be cut off (at locations 926), although neither of them could in some instances also be wrapped around the internal carotid artery IC. Again, the tabs which are used may be stretched and attached to reinforcement strip 910a, as generally described above.

Referring to FIG. 26, in instances where the carotid bifurcation has less of an angle, the structure 900 may be attached using the upper axial attachment tab 906a and be lower transversely angled attachment tab 906d. Attachment tabs 906b and 906c may be cut off, as shown at locations 928 and 930, respectively. In all instances, the elastic nature of backing 902 and the stretchable nature of the electrodes 920 permit the desired conformance and secure mounting of the electrode structure over the carotid sinus. It would be appreciated that these or similar structures would also be useful for mounting electrode structures at other locations in the vascular system.

While the exemplary embodiments have been described in some detail for clarity of understanding and by way of example, a variety of additional modifications, adaptations, and changes may be clear to those of skill in the art. Hence, the scope of the present invention is limited solely by the appended claims.

What is claimed is:

1. An electrode structure comprising:
a elastic backing having a tissue contacting side and an exposed side; and
an electrode disposed on the tissue contacting side;
wherein a drug is sequestered on at least one of the tissue contacting side of the backing and the electrode such that the drug is released into tissue while the backing is engaged against the tissue.

2. The structure of claim 1, wherein the backing is adapted to stretch and contract while the backing is wrapped at least partially around a pulsating tissue structure.

3. The structure of claim 2, wherein the backing comprises an elastic, electrically insulating layer.

4. The structure of claim 3, wherein the backing comprises an elastic layer impregnated with the drug adjacent to the elastic electrically insulating layer.

5. The structure of claim 2, wherein the backing comprises a sheet of elastomer impregnated with the drug.

6. The structure of claim 1, wherein the drug is coated on at least one of a surface of the backing and the electrode.

7. The structure of claim 6, wherein the coating is disposed on the tissue contacting side of the backing.

8. The structure of claim 6, wherein an adhesive impregnated with the drug is disposed on at least one of the tissue contacting side and the electrode.

9. The structure of claim 8, wherein the adhesive attaches the backing to the electrode.

10. The structure of claim 1, wherein the drug comprises a steroid.

11. The structure of claim 1, wherein the electrode is coupled to an implantable pulse generator.

12. The structure of claim 1, wherein the electrode comprises a coil.

13. The structure of claim 1, further comprising at least a second electrode on the tissue contacting side, wherein the drug is sequestered around the first and second electrodes on the tissue contacting side.

14. The structure of claim 1, wherein the electrode comprises a recess, and the sequestered drug is disposed at least partially within the recess.

15. The structure of claim 1, further comprising a core impregnated with the drug, wherein the electrode comprises a coil, and the core is disposed at least partially within the coil.

16. The structure of claim 17, wherein the core is a tube having drug in a central passage.
